(19) United States
(12) Patent Application Publication
(10) Pub. No.: US 2017/0049499 A1
(43) Pub. Date: Feb. 23, 2017

(54) Method of Treating Hemorrhoids

(71) Applicant: Mark Whiteley, Surrey (GB)

(72) Inventor: Mark Whiteley, Surrey (GB)

(21) Appl. No.: 15/240,521

(22) Filed: Aug. 18, 2016

(30) Foreign Application Priority Data
Aug. 19, 2015 (GB) 1514729.1

(51) Int. Cl. A61B 18/14; A61M 5/31; A61N 7/02; A61B 18/18; A61B 18/20

(52) U.S. Cl. CPC A61B 18/14; A61B 18/1815; A61B 18/20; A61N 7/02; A61M 5/31; A61B 2018/00577

(57) ABSTRACT

The invention relates to a method for the treatment of haemorrhoids and the use of said method for embolization and/or ablation of the underlying vein(s) that cause(s) haemorrhoids.

This application claims priority benefit of United Kingdom patent application No. 1514729.1 filed Aug. 19, 205, which is incorporated by reference herein in its entirety.

### FIELD OF THE INVENTION

The invention relates to a method for the treatment of haemorrhoids and the use of said method for embolization and/or ablation of the underlying vein(s) that cause(s) haemorrhoids.

### BACKGROUND OF THE INVENTION

The prevalence of haemorrhoids in the general population in the U.S. is about 5% at any one time and is thought to affect 50% of adults during their lives¹.

Haemorrhoids are swollen blood vessels in or around the anus and rectum. The haemorrhoidal veins are located in the lowest part of the rectum and the anus. Sometimes they swell so that the vein walls become stretched and can be irritated by passing bowel movements. Haemorrhoids are classified into two general categories: internal and external. Internal haemorrhoids lie inside the rectum. They don't usually hurt because there are few pain-sensing nerves in the rectum. Bleeding may be the only sign that they are there. However, they can prolapse, or enlarge and protrude outside the anal sphincter. Prolapsed haemorrhoids may hurt due rubbing from clothing and sitting. They usually recede into the rectum on their own. External haemorrhoids lie within the anus and are often uncomfortable. If an external haemorrhoid prolapses to the outside (usually in the course of passing a stool), you can see and feel it. Blood clots sometimes form within prolapsed external haemorrhoids, causing an extremely painful thrombosis.

Currently, treatments² are classified into one of two groups: non-operative treatment and operative treatment. The former includes:
Injection sclerotherapy—using oily phenol and more recently foam sclerotherapy
Banding—and elastic band is placed around the haemorrhoid to cause it to die and the dead tissue to fall away
Infra-red coagulation
Radiofrequency ablation
Cryotherapy
Laser ablation of Haemorrhoidal artery
Ultrasound ablation
   The latter includes:
Haemorrhoidectomy—surgical removal
Plication
Haemorrhoidal Artery ligation operation (HALO)/Doppler Guided
Haemorrhoidal Artery ligation
Stapled haemorrhoidectomy.

However, all of the current operative methods have one thing in common—the procedures are performed externally, approaching the haemorrhoid from outside and necessitating intervention via the anal canal.

This approach has several disadvantages:p
post-operative pain is present as the treated tissue is in the anal canal which is both sensitive and is also irritated by the passage of faeces,
hospital stay—some of the methods require a hospital stay due to the initial pain,
General anaesthesia or regional anaesthesia—many of the techniques require general anaesthetic or regional anaesthesia due to the pain of the procedure.
Post-operative pain killers—most of these techniques require significant post-operative pain killers,
Inability to treat the underlying cause of the haemorrhoids—treatment of the external bulging vein alone fails to treat the underlying cause,
High recurrence rates—all of the techniques show high recurrence rates particularly in the long term which we suggest is due to the fact that the underlying cause has not been identified and treated in any of the current techniques,
Potential for infection—the anal canal is a dirty area transmitting faeces and so any surgical wound in this area can potentially get infected,
Embarrassment for the patient

There is therefore a need to treat haemorrhoids in a way that addresses their underlying cause with a view to preventing or minimising their re-occurrence and the method described herein has been designed to achieve this objective.

### SUMMARY OF INVENTION

According to a first aspect of the invention there is provided a method for treating and/or preventing haemorrhoids comprising: embolizing and/or ablating at least one pelvic vein particularly the internal iliac vein and/or at least one of its tributaries.

Reference herein to embolization and/or ablation is reference to a, typically minimally invasive, treatment that occludes, or blocks, one or more blood veins or vascular channels.

Reference herein to the internal iliac vein is reference to the iliac vein and, particularly but not exclusively, to its location deep within the pelvis.

Reference herein to at least one of its tributaries is reference to a vessel that joins said vein and whose occlusion will prevent the aberrant flow leading to the formation of said haemorroid(s).

Research published by The Whiteley Clinic³ has shown that haemorrhoids are associated with/caused by venous reflux in the pelvic veins. It appears that the haemorrhoid is merely the bulging end of the venous system of pelvic veins.

Thus, embolization and/or ablation of the specific disease causing vein or veins in the pelvis stops the venous reflux in the vein or veins that is/are causing the haemorrhoids.

Indeed, we have established that haemorrhoids can be treated by pelvic vein embolization and/or ablation of the refluxing pelvic vein and this might be sufficient treatment in itself. In some patients, this might be supplemented with adjuvant treatment, for example, foam sclerotherapy of the haemorrhoids themselves performed at the same time or a selected later time.

Accordingly, in a preferred method of the invention said embolizing and/or ablating of said vein is also accompanied by removal or destruction of the haemorrhoid using any of the methods known in the art selected from the group comprising:
Injection sclerotherapy;
Banding of the haemorrhoid;
Infra-red coagulation;
Radiofrequency ablation;
Cryotherapy;
Laser ablation of Haemorrhoidal artery;
Ultrasound ablation;
Haemorrhoidectomy;
Plication;
Haemorrhoidal Artery ligation operation; and
Stapled haemorrhoidectomy.

Preferably injection sclerotherapy is undertaken using oily phenol or foam sclerotherapy.

Sclerotherapy involves injection of sclerosants into the vein which induces a local inflammatory response resulting in the subsequent shrinking of the vein and its eventual disappearance.

More preferably still Haemorrhoidal Artery ligation is undertaken using (HALO) Doppler Guided Haemorrhoidal Artery ligation.

We have shown that trans-jugular coil embolization of pelvic veins is both safe⁴ and has good results in the medium to long term in terms of permanent venous occlusion of the treated pelvic veins⁵. Accordingly this is practised when working the invention.

Laser and radiofrequency treatment or ablation is a minimally invasive ultrasound-guided technique for treating veins using laser energy (laser) or electrical current alternating at radiofrequency rates (radiofrequency). The process involves passing an optical fibre into a diseased vein, shining laser light into the interior of the vein, destroying it and causing it to be removed from the body by a process of fibrosis. Similarly, radiofrequency ablation of veins involves using the heat generated from a high frequency alternating current to shrink and destroy the vein. The process can be performed under local anaesthesia in an outpatient setting. Indeed, endovenous thermal ablation of these types are used most often to close veins that are damaged by venous reflux disease.

In the case of Endovenous Laser Ablation (EVLA), using ultrasound a needle is passed into the vein under local anaesthetic, and a guide wire then passed up the vein. A long introducer sheath is then passed over the wire. Often, a dilute solution of local anaesthetic is injected around the vein (tumescent anaesthesia) to reduce pain, cool perivenous tissue, and decreases the venous diameter. A fine laser fibre is then passed up inside the sheath, once the guide wire is removed, and when in the correct position, laser energy is delivered to the vein wall which can reach temperatures of 700° C. inside the vein. As the laser energy is delivered, the sheath and fibre are pulled down (usually at about 3-5 mm/s, depending on the power and wavelength) at the right speed thus closing the vein at a precise energy level (typically 60-80 Joules per cm of vein). The amount of delivered energy can be varied by adjusting the pullback speed, the laser energy, or both. Newer techniques have combined sheath and fibre in a single device.

Alternatively, Radio-frequency ablation systems such as the VNUS Closure® FAST™ or Venefit® utilises radio-frequency current to generate heat to seal the vein. A catheter is passed into the vein as in EVLA. The Closure® FAST™ has a 7 cm long end, which heats up to 120° C., and due to its length, eliminates the need for continuous pull-back of energy as required for EVLA permitting delivery of energy to ablate 7 cm segments. Additionally, a 3 cm element is provided to treat smaller vein segments. However, after each segment is treated, the element must still be pulled back and repositioned to treat the next segment. Moreover, the catheter is fairly inflexible and so, whilst suitable for large and regular walled veins, it is less suitable for small side branch veins or for small segments of vein. Further, there is evidence to suggest that it is less suitable for use in the small saphenous vein which is surrounded by numerous large nerves, as heating a large portion can cause nerve damage if not carefully performed.

As with the EVLA and VNUS techniques, RFiTT® is a catheter that is passed into the vein to be treated using a needle under local anaesthetic, and under ultrasound control to ensure it is in exactly the right position. As in the case of the original VNUS Closure® and VNUS Closure® Plus devices, a radio frequency electric current passes between two electrodes at the end of the catheter, heating the vein wall and destroying it. The RFiTT® device passes a great deal of energy very quickly into the vein wall and early results suggest that the procedure can therefore be performed very quickly. However, there is a theoretical concern regarding whether sufficient energy is being passed into the vein wall to permanently destroy the vein as with EVLA, VNUS Closure® and Closure® FAST™. All of these techniques put about 60 to 80 J per centimetre into the vein and have shown complete closure and destruction of the vein at this level. However RFiTT® only puts 20 J per centimetre into the vein, and thus there can be concerns for complete closure of the vein. Recent research from The Whiteley Clinic has shown improved results when the settings are changed to achieve 72 J per centimetre.

There is also mono-polar RFA such as the EVRF® which generates heat in the vein wall in much the same way as the RFiTT® device but using a single electrode on the end of a catheter and using the body's own capacitance as the “second electrode”.

There are also the newer endovenous thermal techniques such as steam vein sclerosis and endovenous microwave ablation.

There are also newer non-thermal techniques that might be used to perform the Hembolize procedure by embolizing the internal iliac veins or its tributaries, including, but not restricted to, mechanochemical ablation (MOCA), cyanoacrylate glue or other materials or devices used for embolizing or occluding blood vessels.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

The present invention will now be described by way of example only with particular reference to the following examples.

### DETAILED DESCRIPTION

### The Hembolize Procedure

The Hembolize procedure is an embolization of the underlying venous reflux in the internal iliac veins and/or tributaries of the internal iliac veins, stopping reflux into the haemorrhoids.

Access to the target veins is via the venous system from a remote location—this can be, but is not restricted to, access via the internal jugular vein, the brachial vein or the femoral vein. The technique for accessing the vein involves passing a device into the vein, guiding it to a position under image control (typically usually x-ray although not restricted to this imaging technique) and then passing embolic material into the vein. Our hembolize procedure currently uses a combination of foam sclerotherapy and metal coil embolization but other embolic material or techniques for venous destruction, such as ablation, may be used.

However, what is new in this technique is that it targets the reflux in the internal iliac vein and/or its tributaries, thus preventing reflux into the haemorrhoids and therefore cutting off the source of the venous reflux that is filling the haemorrhoids.

Therefore in stark contrast to all the other available methods currently used for the treatment of haemorrhoids:

This approach is internal rather than external—the approach being through the venous system and into the target vein where, from within, the underlying cause can be treated rather than attacking the haemorrhoid externally without addressing the underlying cause.

If there is no treatment to the haemorrhoid itself and no procedure performed across the anal canal, there is either no or minimal post-operative pain.

There is no cut nor healing tissue anywhere in the anal canal and therefore the passing of faeces causes no discomfort in the post-operative period.

The procedure is performed under local anaesthetic and therefore is a walk-in walkout procedure with no requirement for any hospital stay, general anaesthesia or regional anaesthesia.

There is no need for any post-operative pain killers.

As the underlying cause of the haemorrhoids has been treated there will be a lower recurrence rate.

There is minimal embarrassment to the patient as the operation is performed through the chosen access point, for example, the neck (via the internal jugular vein).

### First Case:

The first case of Hembolize was performed at the Whiteley clinic in London on 28 Mar. 2015. The patient was male. He had previously had operative haemorrhoidectomy, injections and most recently a new technique using a probe, from externally, through the haemorrhoid to try and ablate the vein at the neck of the haemorrhoid. Unfortunately this had caused damage to the prostate and blood in the semen.

He had large haemorrhoids which were bleeding and uncomfortable. He consented to Hembolize as the alternative was a further haemorrhoidectomy.

This was performed via a right internal jugular approach and x-ray control on 28 Mar. 2015. It was performed under local anaesthetic as a walk-in walkout procedure. No post-operative pain killers were given or taken.

Since that time, his bleeding at passing stool has gone from every time to very rarely. His haemorrhoids have been shrinking weekly since the procedure, and the discomfort has been improving. He has been offered adjuvant injections but has refused them as the haemorrhoids are continuing to wither away following Hembolize.

### Second Patient:

A male patient with haemorrhoids that were bleeding, protruding and causing severe discomfort underwent Hembolize on the eighth of August 2015 at the Whiteley clinic in Bond Street London.

Once again this was performed as a walk-in walkout procedure under local anaesthetic via a right internal jugular approach and under x-ray control. The patient reported on 11 August that his three major symptoms had completely resolved and he was delighted. He had had no further bleeding when passing stools, the haemorrhoids are starting to shrink and the discomfort had disappeared.

### REFERENCES

1—Lorenzo-Rivero S. Hemorrhoids: diagnosis and current management. Am Surg. 2009 August;75 (8):635-42.
2—Lohsiriwat V. Hemorrhoids: From basic pathophysiology to clinical management. World J Gastroenterol 2012 May 7; 18 (17): 2009-2017
3—Holdstock J M, Dos Santos S J, Harrison C C, Price B A, Whiteley M S.

Haemorrhoids are associated with internal iliac vein reflux in up to one-third of women presenting with varicose veins associated with pelvic vein reflux. Phlebology. 2015 March;30 (2):133-9
4—Ratnam L A, Marsh P, Holdstock J M, Harrison C S, Hussain F F, Whiteley M S, Lopez A. Pelvic vein embolisation in the management of varicose veins. Cardiovasc Intervent Radiol. 2008 November-December; 31 (6):1159-64.
5—Dos Santos S J, Holdstock J M, Harrison C C, Whiteley M S. Long-term results of transjugular coil embolisation for pelvic vein reflux—Results of the abolition of venous reflux at 6-8 years. Phlebology 2015—In Press.

1. A method for treating and/or preventing haemorrhoids comprising:
embolizing and/or ablating at least one pelvic vein and/or at least one of its tributaries.

2. The method according to claim 1, wherein said vein is an internal iliac vein or a tributary thereof.

3. The method according to claim 1, wherein said embolizing occludes, or blocks, said vein.

4. The method according to claim 1, wherein said embolizing and/or ablating involves the use of one of the following: laser or radiofrequency ablation; coil embolization; sclerotherapy; glue; clarivein MOCA; Endovenous Laser Ablation (EVLA); VNUS Closure® FAST™ or Venefit®; RFiTT®; EVRF®; steam vein sclerosis; and endovenous microwave ablation or any other device or substance used for embolizing, ablating or occluding blood vessels.

5. The method according to claim 1, wherein said method further involves treatment or removal of the haemorrhoids.

6. The method according to claim 5, wherein said treatment or removal of the haemorrhoids involves sclerotherapy of the haemorrhoid.

7. The method according to claim 6, wherein said treatment or removal of the haemorrhoids is performed at the same time as said embolizing of said vein.

8. The method according to claim 5, wherein said treatment or removal of the haemorrhoids is undertaken using any one or more methods selected from the group comprising: injection sclerotherapy; banding of the haemorrhoid; infra-red coagulation; radiofrequency ablation; cryotherapy; laser ablation of Haemorrhoidal artery; ultrasound ablation; haemorrhoidectomy; plication; Haemorrhoidal Artery ligation operation; and stapled haemorrhoidectomy.

9. The method according to claim 8, wherein injection sclerotherapy is undertaken using oily phenol or foam sclerotherapy.

10. The method according to claim 8, wherein Haemorrhoidal Artery ligation is undertaken using (HALO) Doppler Guided Haemorrhoidal Artery ligation.

11. The method according to claim 1, wherein trans-jugular coil embolization of said pelvic vein is performed.
